(19) United States
(12) Patent Application Publication
(10) Pub. No.: US 2014/0156011 A1
(43) Pub. Date: Jun. 5, 2014

(54) Modified Reverse Joint and Revision Prosthesis

(71) Applicant: Hip Innovation Techonology LLC, Boca Raton, FL (US)

(72) Inventor: Zafer Termanini, Boca Raton, FL (US)

(21) Appl. No.: 13/895,712

(22) Filed: May 16, 2013

Related U.S. Application Data
(63) Continuation-in-part of application No. 13/024,381, filed on Feb. 10, 2011, now Pat. No. 8,540,779.
Application No. 13/024,381 is a continuation-in-part of application No. 12/799,609, filed on Apr. 28, 2010, now Pat. No. 8,313,531.
(60) Provisional application No. 61/339,680, filed on Mar. 8, 2010.

(51) Int. Cl. A61F 2/34; A61F 2/40

(52) U.S. Cl. CPC A61F 2/34; A61F 2/4081
USPC 623/19.12; 623/22.16

(57) ABSTRACT

A reverse joint prosthesis to replace a “ball and socket” joint such as a hip or shoulder. In the case of a hip, the prosthesis comprises an acetabular cup for implanting in an acetabular socket. The acetabular cup is secured to the acetabular socket of the pelvis. The acetabular cup has a stem extending from the center of a concave portion thereof and an acetabular ball is attached to the stem. A femoral implant is provided with a femoral cup attached to the proximal end thereof. The femoral cup has a stem which can be constructed in a modular fashion with several stem lengths to accommodate different size patients. After implantation of the acetabular cup and ball and the femoral cup, the members are assembled together so that the femoral cup can articulate on the acetabular ball. The prosthesis of the invention has substantially reduced likelihood of dislocation during extreme ranges of motion.

### CROSS REFERENCE TO RELATED APPLICATION

This application is a continuation-in-part of application Ser. No. 13/024,381, filed Feb. 10, 2011, which in turn is a continuation-in-part of application Ser. No. 12/799,609 filed Apr. 28, 2010, and issued on Nov. 20, 2012 as U.S. Pat. No. 8,313,531 B2, which claims the benefit of U.S. Provisional Patent Application Ser. No. 61/339,680 filed on Mar. 8, 2010 and entitled “INTERLOCKING REVERSE HIP PROSTHESIS,” and the entirety of both are incorporated herein by reference.

### BACKGROUND OF THE INVENTION

1. Field of the Invention

The present invention relates generally to joint prostheses and more specifically to modified reverse joint prostheses allowing increased range of motion and stability during excessive ranges of motion.

2. Description of the Prior Art

It can be appreciated that several joint implants have been in use for years. For example, conventional hip implants comprise a femoral component having an articulating femoral ball attached to a femoral stem. The femoral stem is inserted into the medullary canal of the femur after preparation and reaming using appropriate reamers by the operating surgeon. The stem can be secured with bone cement or press fit. An acetabular component having the shape of a cup is inserted into an acetabular socket after preparation and appropriate reaming and secured with cancellous screws through holes in the cup. It can also be secured with bone cement or press fit or a combination thereof.

The acetabular cup is metallic and it is internally lined with high-density polyethylene or ceramic. Said lining is secured into the acetabular cup by a press-fit mechanism.

The main problem with conventional hip implants is the instability of the prosthesis at extreme ranges of motion, thereby allowing the femoral ball to dislodge and dislocate. Prior art teaches constrained and preassembled ball and cup devices or devices wherein the ball and cup members are implanted separately whereupon the ball element is forced into a resilient opening in the cup and thereafter held in place by the resilient material. Other constrained acetabular cups may include a locking ring such as the one described by Albertorio et al. U.S. Pat. No. 6,527,808. In the case of cup elements having retaining rings, the ball member is forcefully inserted into the cup after the two elements are implanted. This constitutes a weak link where forces exerted on the prosthesis by ambulatory motion may exceed the forces used to assemble the implant thereby causing the ball to be separated from the cup.

While these devices may be suitable for the particulating purpose which they address, they do not provide an interlocking mechanism as in the reverse joint prosthesis design of the present invention. The very nature of applicant's design allows increased range of motion and increased stability at extreme ranges of motion thereby reducing the risk of dislocation.

In these respects, the interlocking reverse joint prosthesis according to the present invention substantially departs from the conventional concepts and designs of the prior art because, in the case of a hip implant, for example, the articulating femoral ball of the prior art is replaced with an articulating femoral cup and the acetabular cup is provided with an acetabular ball. Thus an apparatus is provided which is primarily developed for the purpose of reducing the risk of dislocation of joint implants at extreme ranges of motion.

Furthermore, when the articulating surface of the femoral cup of the invention is articulating on the acetabular ball, it is fully in contact 100% of the time with the surface of the acetabular ball. It is dear that this will improve tribology because the weight bearing distribution is improved on the articulating surfaces, thus decreasing the wear of the surfaces in contact and reducing the risk of wear particles being released in the joint. The later, being very detrimental to the proper function of the joint.

In another embodiment of the invention, the edge of the acetabular cup is notched to permit the femoral cup to articulate at a greater angle than would be permitted without the notch. While the permitted range of motion without dislocation is substantially improved by the present invention as compared with the prior art, the notched acetabular cup permits an even greater range of motion. In a preferred embodiment, the femoral cup is also notched when the acetabular cup is notched and this permits yet a greater range of motion.

The invention is described for the most part with reference to a hip prosthesis for convenience of the description. However, the invention is not limited to a prosthesis for the hip and it can be adapted for use with other joints without departing from the basic principles described herein. For example, the prosthesis can be used in the shoulder and in other mammalian “ball and socket” type joints. When the implant of the invention is a shoulder joint prosthesis, a glenoid cup is firmly attached to the concave surface of the glenoid fossa. The glenoid cup has a glenoid cup stem and a glenoid ball firmly affixed thereto. A humeral cup articulates on the glenoid ball and the numeral cup has a stem like protrusion which is firmly attached to a humeral stem to be inserted into the medullary canal of the proximal humerus.

### SUMMARY OF THE INVENTION

The present invention provides a new interlocking reverse joint prosthesis construction wherein, in the case of a hip for example, an acetabular ball is solidly and concentrically attached to a central protrusion or stem of an acetabular cup via Morse taper. A metallic acetabular cup is used in the preferred embodiment. A femoral cup, also referred to herein as a hemispherical femoral cup or an articulating femoral cup, is solidly attached to a femoral implant preferably by means of a Morse taper. Other means of attachment known to those in the art can be used. And whenever a Morse taper is referred to herein, it is intended to describe a preferred embodiment. The Morse taper can be replaced by other suitable means of attachment as will be apparent to those having skill in the art.

The acetabular cup is implanted in an acetabular socket constructed by the surgeon in the pelvic bone to which it is firmly secured, preferably by one or more fasteners through one or more openings in the acetabular cup. It can also be secured with bone cement or press fit or a combination thereof, in which case the fasteners and holes for the fasteners are optional. When fasteners are used, they can be cancellous screws or biocompatible resorbable studs of variable number. The femoral implant is then inserted and impacted into the femoral medullary canal which has been prepared and hollowed by the surgeon using appropriate reamers. During ambulation, the articulating femoral cup edge or lip will glide conformably and concentrically into a space or gap located between the acetabular ball and the acetabular cup. As will be apparent to those having skill in the art, the geometrical configuration of applicant's invention makes it very difficult for the femoral cup to dislocate when the range of motion increases since it becomes constrained in the gap between the acetabular cup and the acetabular ball. When the acetabular cup is optionally notched, the range of motion increases further.

As noted above, when the articulating surface of the femoral cup is articulating on the acetabular ball, it is fully in contact at all times with the surface of the acetabular ball. This improves the weight distribution, decreases the wear of the surfaces in contact and reduces the risk of wear particles being released in the joint.

In an optional embodiment of the invention, applicant has addressed the rare possibility that soft tissue may get lodged in the implant in the space between the acetabular cup and the acetabular ball. A protective sheath can be used to avoid this possibility. As discussed in more detail below, the sheath is disposed in the space between the acetabular cup and the acetabular ball and is allowed to glide freely therein.

In another optional embodiment of the invention, the acetabular cup is notched to permit the femoral cup to articulate at a greater angle than would be permitted without the notch. When the acetabular cup is notched, another option is to notch the femoral cup to permit an even greater range of motion.

There has thus been outlined the more important features of the invention in order that the detailed description may be better understood, and so that the present contribution to the art may be better appreciated. A novel feature of this invention is that the location of the articulating surfaces of the joint, namely the ball and socket, is reversed. This results in a new reverse joint implant which is not anticipated, rendered obvious, suggested or even implied by any prior joint prosthesis when considered alone or in any combination,

In this respect, before explaining at least one embodiment of the invention in detail, it is to be understood that the invention is not committed in its application to the details of construction and the arrangements of the components set forth in the following description or illustrated in the drawings. The invention is capable of other embodiments and of being practiced and carried out in various ways as will be apparent from the description herein to those having skill in the art. Also, it is to be understood that the terms employed herein are for the purpose of the description and should not be regarded as limiting.

To the accomplishment of the above and related objects, this invention may be embodied in the form illustrated in the accompanying drawings. However, the drawings are elicited only and changes may be made into any specific construction illustrated without departing from the principles of the invention.

### BRIEF DESCRIPTION OF THE DRAWINGS

Various other objects, features and advantages of the present invention will become fully appreciated as the same becomes better understood when considered in conjunction with the accompanying drawings, in which like reference characters designate the same or similar elements throughout the several views, and wherein:

FIG. 1 is a perspective view of the interlocking reverse hip prosthesis of the invention.

FIG. 2 is a section view of the interlocking reverse hip prosthesis.

FIG. 3 is a section view of the interlocking reverse hip prosthesis in extension and external rotation.

FIG. 4 is a section view of the interlocking reverse hip prosthesis in flexion and internal rotation.

FIG. 5 is a perspective view of the prosthesis of the invention illustrating an optional soft tissue protective sheath.

FIG. 5A is a perspective view of the protective sheath by itself.

FIG. 6 is a section view of the embodiment of FIG. 5.

FIG. 7 is a section view illustrating a femoral cup having a recess instead of a stem for connection to a femoral implant.

FIG. 8 is a section view of the embodiment of FIG. 7 which has been articulated to an extreme position.

FIG. 9 is a &de elevation view of a notched acetabular cup.

FIG. 10 is a top view of the notched acetabular cup of FIG. 9.

FIG. 11 is a side elevation view of an acetabular cup without a notch illustrating the maximum articulation of the femoral cup therein.

FIG. 12 is a side elevation view of a notched acetabular cup illustrating the increased maximum articulation of the femoral cup therein.

FIG. 13 is a section view illustrating a femoral cup in a position of maximum articulation in a notched acetabular cup.

FIG. 14 illustrates in section a notched acetabular cup implanted in a pelvic bone.

FIG. 15 is a perspective view of a notched femoral cup.

### DESCRIPTION OF THE PREFERRED EMBODIMENTS

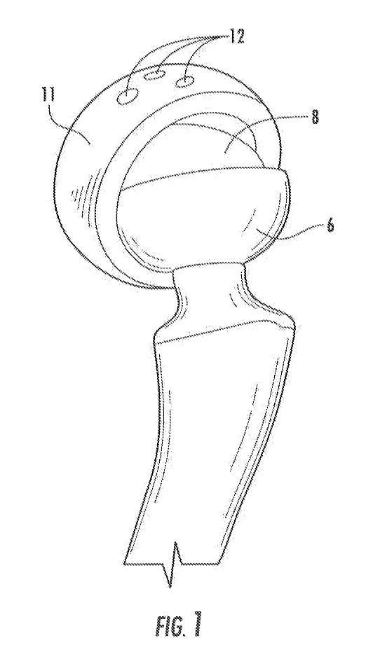

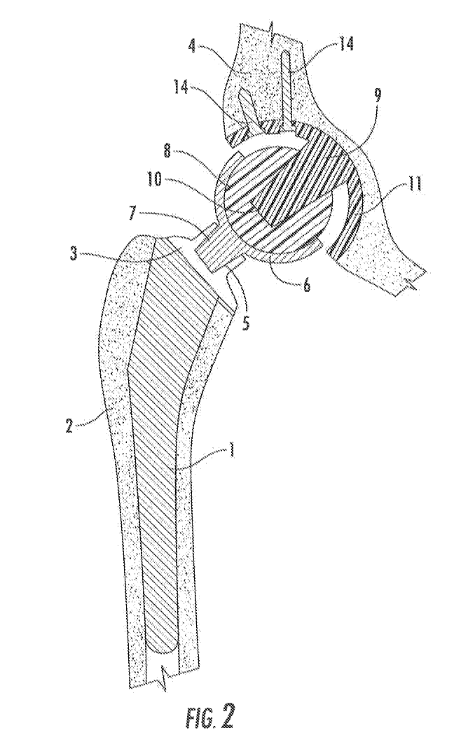

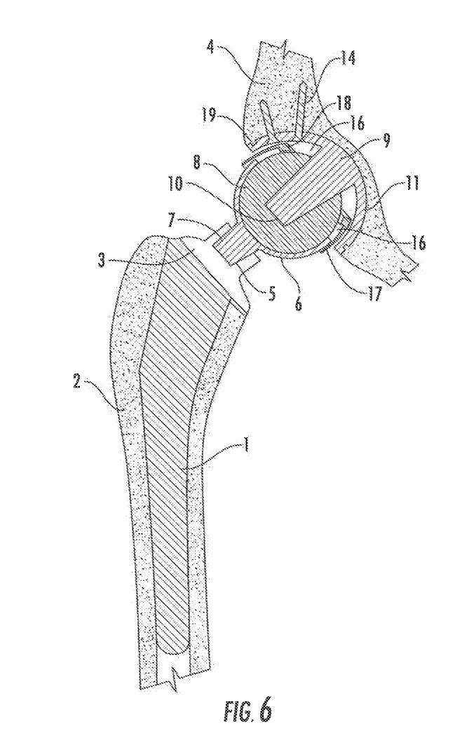

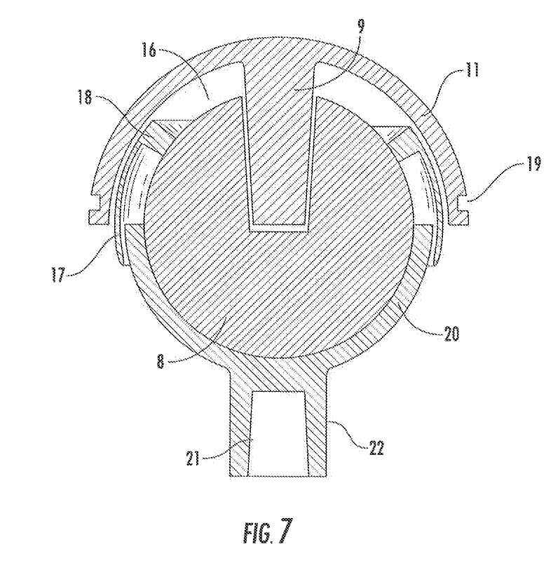

Turning now to the drawings, in which the reference characters denote the same or similar elements throughout the several views, FIGS. 1-4 illustrate an interlocking reverse hip prosthesis, which comprises an acetabular cup (11) having a smooth concave surface and a non-articulating surface. The non-articulating surface, which can optionally be convex, abuts a socket in the pelvic bone and is firmly affixed thereto when the acetabular cup is implanted in a patient. The non-articulating surface preferably provides a porous surface with multiple asperities and micro-voids to allow bone ingrowth. Furthermore, the acetabular cup (11) provides one or more optional holes (12) at different locations for the purpose of using one or more optional fasteners (14). The fasteners (14) can be screws or resorbable nonmetallic and biocompatible studs of different diameters and lengths. The studs, which can be called orthobiologic resorbable studs, will secure the acetabular cup (11) during the initial phase of bone ingrowth and will resorb within one year, being replaced by newly generated bone and become part of the host pelvic bone. During that period, the acetabular cup (11) becomes solidly attached to the acetabular socket in the pelvic bone (4) by bone ingrowth. The acetabular cup (11) has a concave hemispherical surface in which a large acetabular cup stem (9) is disposed. The acetabular cup stem (9) has a male Morse taper for assembly to the acetabular ball (8) by means of the acetabular ball recess (10) which has a female Morse taper. Referring to FIGS. 2-4 and 6, the femoral cup (6) has a femoral cup stem (7) with a male Morse taper while the femoral implant (1) has a cooperating femoral implant recess (5) having a female Morse taper located in neck (3). FIGS. 7 and 8 illustrate a femoral cup (20) with a neck (22) and a femoral cup recess (21) having a female Morse taper. This cooperates with a femoral implant stern having a male Morse taper (not shown). In a preferred embodiment, a modular system is used in a kit according to the invention wherein the femoral cup stern (7) or neck (22) can come in different lengths to accommodate the sizing needs of a patient. Therefore, in a kit of the invention, two or more femoral cups are provided having different stem lengths. In a less preferred embodiment, the length of the neck (3) of femoral implant (1), or the femoral implant stem (not shown) can also have various lengths to accommodate patient sizing needs and in a kit comprising this embodiment two or more neck or stem components having different lengths will be included. Other variations on the design to meet different sizing needs will be apparent to those having skill in the art.

An important advantage of the present invention is that the greater the interdigitation the more stability of the implant as opposed to conventional ball and socket hip implants, where increased range of motion is usually associated with increased risk of dislocation.

Referring to FIG. 2, the proximal femoral bone (2) is reamed in the usual fashion to accept a femoral implant (1) that can be cemented or press fitted in the femoral medullary canal. The acetabular socket in the pelvic bone (4) is reamed to the appropriate size to accept the acetabular cup (11), which is impacted for press fit at the correct angle of inclination and anteversion. Fasteners (14) in the form of fixation screws or biocompatible resorbable studs are then inserted in place to secure the acetabular cup (11). The acetabular ball (8) is then affixed onto the acetabular cup stem (9). FIG. 2 also illustrates center line (C-C). In the position shown, the center line (C-C) passes through the center of the acetabular cup (11), the longitudinal center line of the acetabular cup stem (9), the center of acetabular ball (8), the longitudinal center line of femoral cup stem (7) and the longitudinal center line of femoral implant recess (5). Obviously, when the femoral cup is articulated on the ball the center line associated with the femoral components will not be colinear with the center line of the acetabular components. The line is simply illustrated in this way for convenience.

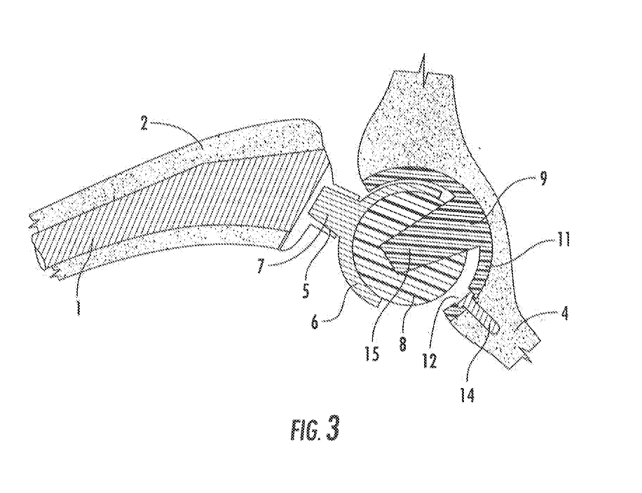

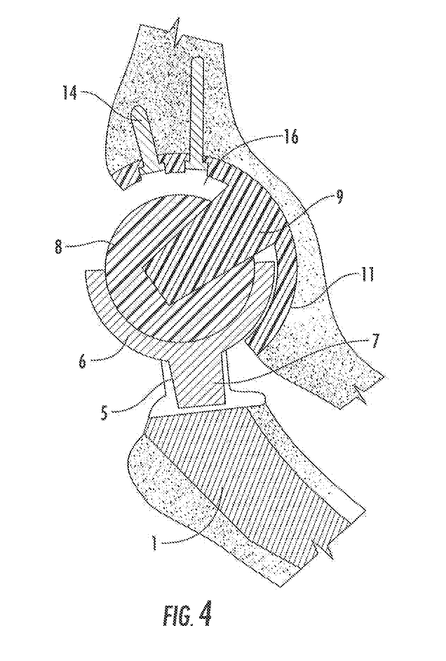

Referring to FIGS. 3, 4 and 8, when the femoral cup (6) or (20) articulates on the acetabular ball (8), the edges of the femoral cup (6) or (20) move into and out of the hemispherical space or gap (16) and the articulating surface of the femoral cup (6) or (20) maintains the same area of contact with the acetabular ball (8) over the entire range of motion. In other words, 100% of the articulating contact area of the femoral cup (6) or (20) is maintained over the entire range of motion when the femoral cup is articulating on the surface of the acetabular ball. FIG. 3 illustrates the prosthesis of the invention in extension and external rotation. FIG. 4 illustrates the prosthesis in flexion and internal rotation and FIG. 8 illustrates an extreme articulation position of the femoral cup (20) on the acetabular ball (8).

It is well known that certain types of movement or positions of the femur relative to the acetabulum cause increased risk of dislocation of the “ball and socket” particularly in the case of conventional prior art hip implants. For example, when an individual crosses his or her legs, lays on his or her side while sleeping or extends his or her leg to an extreme position such as during a ballet performance. When this occurs with the hip implant of the present invention, the femoral cup (6) or (20) is constrained in the hemispherical space or gap (16) between the acetabular ball (8) and the acetabular cup (11) and the convex surface of the femoral cup may come into contact with and articulate on the concave surface of the acetabular cup.

In one embodiment, the articulating surface of the femoral cup (6) or (20) contains a high molecular weight polyethylene lining of varying thickness, but no less than 4 mm. In a different embodiment the lining could be porcelain, ceramic or a metallic alloy.

An important feature of the present invention is the ability to place acetabular ball (8) in a position that minimizes or eliminates tortional forces on the acetabular cup and acetabular stem. This is illustrated in FIG. 3 wherein the acetabular ball (8) is affixed on acetabular cup stem (9) in a position wherein the equatorial plane (P-P) of the acetabular cup passes through the center (15) of the acetabular ball.

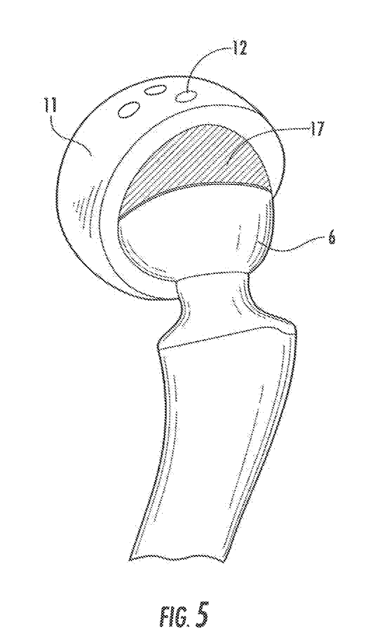

An optional embodiment of the invention illustrated in FIGS. 5-8 adds a soft tissue protective sheath (17) to the embodiments described above. The protective sheath, also illustrated by itself in perspective in FIG. 5A, addresses the rare cases wherein soft tissue might get lodged in the gap (16) as a result of articulation of the prosthetic joint of the invention.

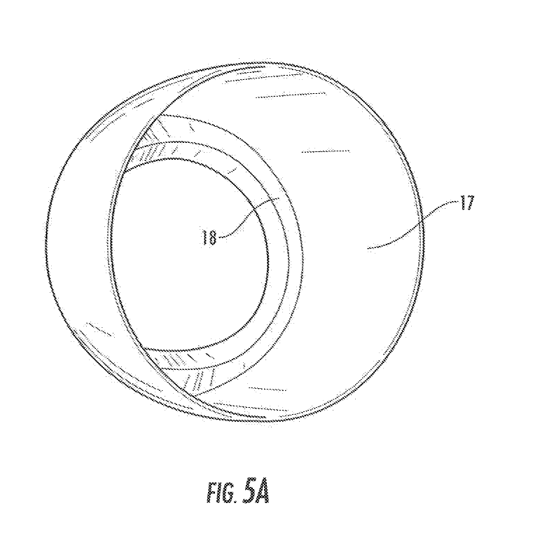

Referring to FIGS. 6-8, the sheath (17) extends beyond the circular outer edge of the acetabular cup (11) and has a retaining ring (18). The sheath (17) is installed by placing it into the acetabular cup (11) before the acetabular ball (8) is installed. The sheath (17) can have a solid surface as illustrated or it can be perforated with holes, slots or the like having the same or different shapes and dimensions as may be desired.

As can be seen from FIGS. 6-8, the sheath (17) is allowed to move freely within the space (16), restrained only by the acetabular ball (8) and acetabular cup stem (9). The sheath (17) also is moved by contact of retaining ring (18) with the outer edge of femoral cup (6) or (20). The retaining ring (18) will, for example, contact the outer edge of femoral cup (6) or (20) particulatingly in positions of extreme articulation of the prosthetic joint as illustrated in FIG. 8.

In another embodiment of the present invention, also illustrated in FIGS. 6-8, the acetabular cup (11) is designed for use in revision surgery of the hip. Revisions are surgical procedures where the existing implant is removed. This most frequently requires removal of the acetabular cup and it is associated with a high level of morbidity. The removal of a previously implanted acetabular cup may be quite difficult surgically, especially when the cup has metallic beads for bone ingrowth. In these cases, the removal is also associated with iatrogenic bone loss leading to difficulty in inserting another conventional acetabular cup.

There are instances where the acetabular cup was not implanted correctly or where the lining of the existing implant becomes worn out and needs to be replaced. Recurrent dislocations of the hip implant are usually secondary to surgical misplacement of a conventional acetabular cup. For example, if during the initial procedure, the cup was placed either too vertical or retroverted (e.g., facing backward instead of forward).

To remedy the above cited complication, there are times where the surgeon simply cements a conventional revision cup into the previously implanted acetabular cup using conventional bone cement. However, problems arise if the initial position of the previously implanted acetabular cup is too vertical or retroverted, preventing a conventional revision cup from being glued in the previously implanted acetabular cup. As a result, removal of the previously implanted acetabular cup becomes necessary—entailing significant risk and possible morbidity to the patient as described above.

Another important advantage of the present invention is that the angle of inclination and retroversion are not critical since the interlocking mechanism of applicant's implant will compensate for the misalignment of a previously implanted acetabular cup.

The acetabular cup (11) of applicant's interlocking reverse hip prosthesis can optionally be provided with a thin circumferential groove (19) located in proximity of the equatorial plane of said cup as illustrated in FIGS. 6-8.

In revision surgery using the hip prosthesis of applicant's invention, the plastic polyethylene insert of the previously implanted acetabular cup is removed. The circumferential groove (19) of the interlocking reverse hip prosthesis will host a retaining “o-ring” of the previously implanted acetabular cup being revised thereby providing solid fixation of applicant's revision interlocking reverse hip prosthesis to the previously implanted acetabular cup.

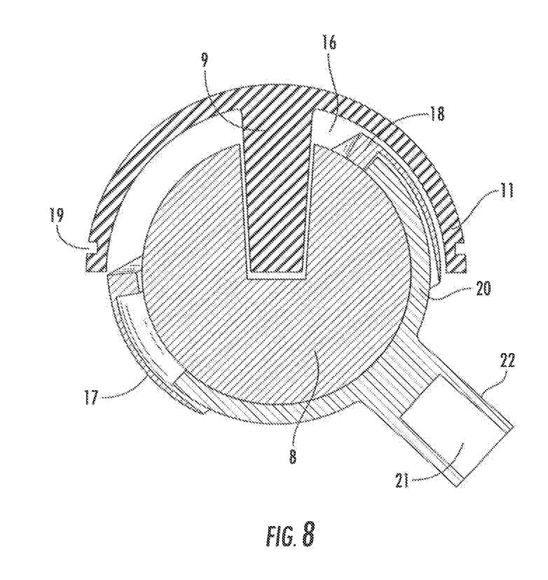

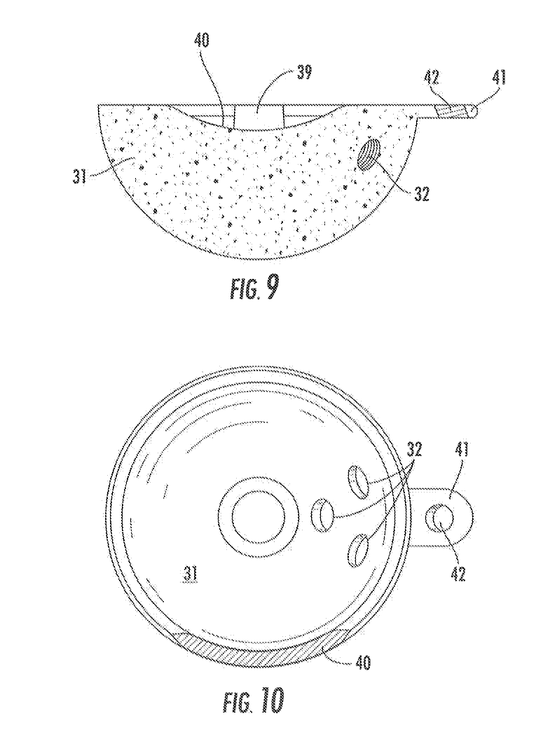

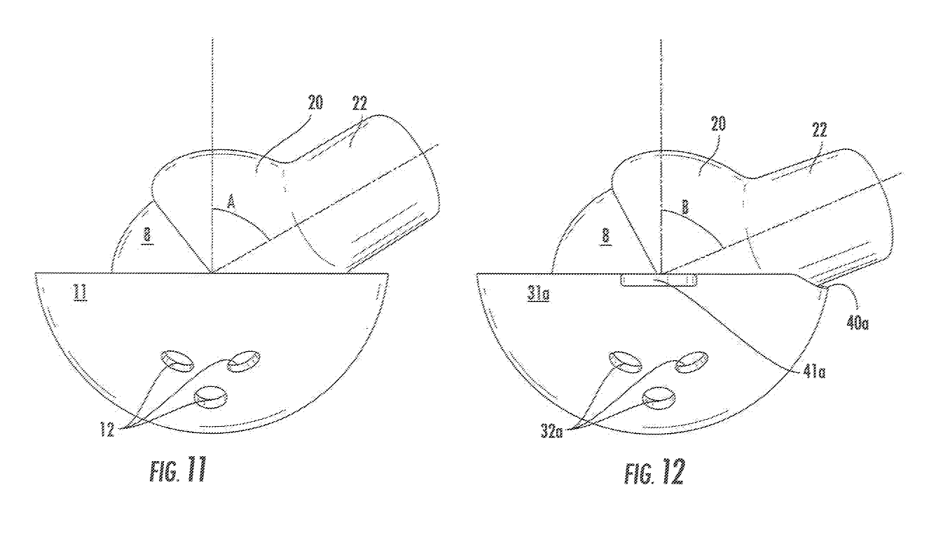

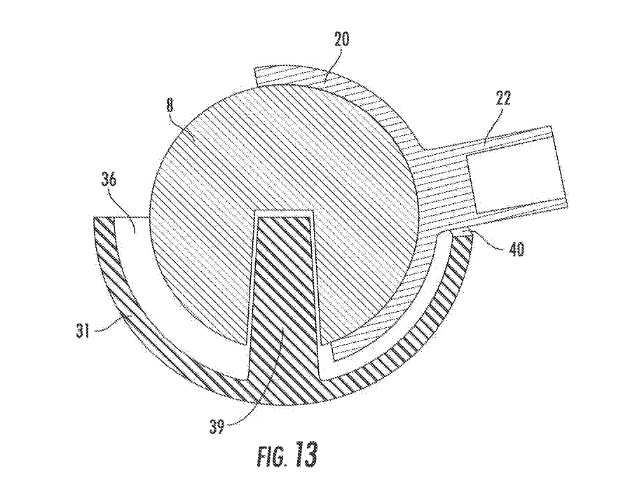

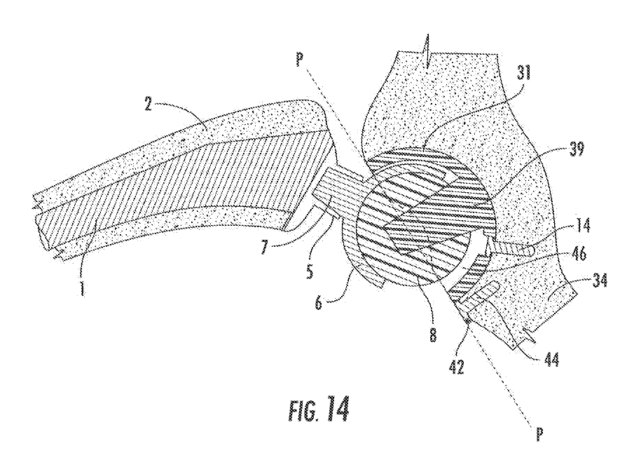

While the range of motion permitted without dislocation by the prosthesis described and illustrated herein with respect to FIGS. 1-8 is substantially improved over the prior art, an optional notched acetabular cup embodiment permits an even greater range of motion. This is illustrated by FIGS. 11-13 wherein FIG. 11 illustrates the range of motion permitted by the embodiment of FIGS. 1-8, FIG. 12 illustrates the range of motion permitted by the notched acetabular cup (31) and FIG. 13 illustrates in section a femoral cup in a position of maximum articulation in a notched acetabular cup. Thus, the angle B in FIG. 12 is greater than angle A in FIG. 11. For example, angle A may be 60° and angle B may be 70° or more. FIGS. 9, 10 and 14 further illustrate the optional embodiment of the prosthesis of the invention wherein acetabular cup (31) has a notch (40) in the circumferential outer edge thereof and the acetabular cup (31a) has a notch (40a). The notches (40) and (40a) each are located at positions on the respective acetabular cups (31) and (31a) that will permit maximum inflection when the prosthesis of the invention is implanted in a patient. Inflection refers to the position wherein a patient is sitting down or squatting. Thus, the acetabular cup (31) as illustrated herein would be implanted in a right hip and acetabular cup (31a) would be implanted in a left hip. The width and depth of the notch (40) or (40a) can be varied, as will be apparent to those having skill in the art, consistent with an objective of the invention to reduce the risk of dislocation.

Except for the notch (40), acetabular cup (31) comprises the same elements as acetabular cup (11). The cup (31) has a stem (39) affixed firmly therein and holes (32) for fasteners. The holes (32) can optionally be threaded. An optional tab (41) with an optionally threaded hole (42) for a fastener can be provided on any of the acetabular cups of the invention and it is illustrated herein on acetabular cup (31).

FIG. 14 illustrates in section the prosthesis of the invention implanted in the pelvic bone (34) wherein the acetabular cup (31) is provided with optional tab (41) and a screw (44) screwed into pelvic bone (34) through hole (42). Acetabular cup stem (39) is firmly affixed to, or is a unitary part of, acetabular cup (31) and gap (46) between acetabular ball (8) and the concave portion of acetabular cup (31) allows femoral cup (6) to articulate therein.

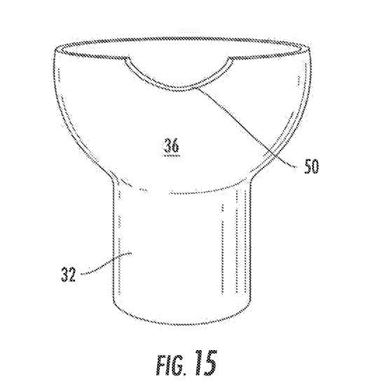

FIG. 15 is a perspective view of an optionally notched femoral cup (36). The notch (50), located on the circumferential outer edge of femoral cup (36), permits a greater range of motion when cup (36) is used in combination with notched acetabular cup (31) because the notch (50) will allow the femoral cup (36) to articulate more closely to the stem (39). Thus, the stem (39) will not impede the articulation of cup (36) at the most extreme range of motion, especially in the case where notch (40) or (40a) is deep. As with the notches (40) and (40a) of respective acetabular cups (31) and (31a), the notch (50) can be varied in its width and depth. The location of notch (50) is coordinated with the location of notch (40) or (40a), as, explained above, in order to maximize the range of motion. When the neck (32) of femoral cup (36) is adjacent the lowest point of notch (40) or (40a), the stem (39) of acetabular cup (31) or (31a) preferably should be adjacent the lowest point of notch (50).

The components of the reverse hip prosthesis of the invention are made from biocompatible materials commonly used in the art and suitable materials will be apparent to those skilled in the art based upon the disclosures herein. Metals or metallic alloys such as titanium or cobalt chrome are suitable. For some components, such as the acetabular baa, metals or ceramics can be used. High density polyethylene is also suitable for some components, for example the protective sheath or an optional lining for the concave portion of the femoral cup. Other biocompatible materials or combinations thereof can be used for various components as will be apparent to those having skill in the art.

The dimensions of the various components of the reverse hip prosthesis of the invention can be readily determined by those skilled in the art based upon the disclosures herein. For a hemispherical acetabular cup, an outer diameter from about 35 millimeters (mm) to about 65 mm will be suitable for most applications. The spherical acetabular ball should have a diameter from about 28 mm to about 45 mm. The diameter of the acetabular ball should be from about 7 mm to about 12 mm smaller than the inner diameter of the acetabular cup, thus creating a hemispherical space or gap having a width form about 7 mm to about 12 mm to allow articulation of the femoral cup therein. Of course, the concave, hemispherical, articulating surface of the femoral cup will be sized compatibly with the acetabular ball to allow for smooth articulation. Highly polished cobalt chrome is an excellent material for the articulating surface of the femoral cup but other materials such as biocompatible metallic alloys can be used. The femoral cup also may contain a lining fabricated from high-density polyethylene, ceramic or biocompatible metallic alloys.

It is therefore an object of the present invention to provide a new and improved interlocking and restrained reverse hip prosthesis system, where two conventional articulating surfaces of the hip joint are reversed and constrained at extreme ranges of motion in a manner that significantly reduces the risk of dislocation. The system described in the present invention has all of the advantages of the prior art designs, none of the disadvantages, and numerous improvements over the prior art, particularly in respect of remarkably increased range of motion without dislocation and reduced risk of wear particles being released into the joint.

Another embodiment of the present invention is directed to a prosthesis for a shoulder joint. The first component includes an anchoring glenoid plate or glenoid cup firmly attached to the concave surface of the glenoid fossa. The glenoid cup having a glenoid cup stem and a glenoid ball firmly affixed thereto. The second component being a hemispherical humeral cup having a stem like protrusion which is firmly attached via Morse taper to a humeral stem to be inserted into the medullary canal of the proximal humerus.

The reverse shoulder prosthesis of the invention comprises a unitary glenoid cup having a non-articulating surface, which optionally can be convex, for firm, non-articulating attachment to a glenoid fossa. A concave surface is located opposite to the non-articulating surface and the concave surface has a glenoid cup stem firmly affixed therein and projecting outwardly therefrom. A glenoid ball having a surface is firmly affixed to the glenoid cup stem. The concave surface of the glenoid cup and the surface of the glenoid ball are spaced from one another, thereby defining a gap therebetween.

The reverse shoulder prosthesis further comprises a humeral stem for implantation in a medullary canal of a proximal end of a humerus. A humeral cup is firmly affixed to a proximal end of the humeral stem. The humeral cup is sized for articulation in the gap, such that the humeral cup has a concave surface sized for articulation on the surface of the humeral ball and a convex surface, opposite the concave surface of the humeral cup, sized for articulation on the concave surface of the glenoid cup. The gap is sized and configured to permit said articulations while constraining the humeral cup within the gap throughout an entire range of said articulations of the humeral cup as it articulates within the gap, thereby reducing the risk of dislocation.

It should be apparent to one skilled in the art when referring to the drawings herein that there is an equivalence of the elements of the hip prosthesis and the shoulder prosthesis of the invention. Thus, among others, the glenoid cup is equivalent to the acetabular cup, the glenoid cup stem is equivalent to the acetabular cup stem, the glenoid ball is equivalent to the acetabular ball, the humeral stem is equivalent to the femoral stem and the humeral cup is equivalent to the femoral cup.

With respect to the above description then, it is to be realized that the optimum dimensional relationships for the parts of the invention, to include variations in size, materials, shape, form, function and manner of operation, assembly and use, are deemed readily apparent to those skilled in the art, and all equivalent relationships to those illustrated in the drawings and described in the specification are intended to be encompassed by the present invention. Therefore, the foregoing is considered as illustrative only of the principles of the invention. Further, since numerous modifications and changes will readily occur to those skilled in the art, it is not desired to limit the invention to the exact construction and operation shown and described, and accordingly, all suitable modifications and equivalents may be resorted to, falling within the scope of the invention.

What is claimed is:

1. A reverse hip prosthesis comprising
a unitary acetabular cup having an outer non-articulating surface adapted for attachment to an acetabular socket in a pelvic bone and a concave surface located opposite to the non-articulating surface, the concave surface having an acetabular cup stem firmly affixed therein and projecting outwardly therefrom,
an acetabular ball firmly affixed to the acetabular cup stem, the acetabular ball having a surface,
the concave surface of the acetabular cup and the surface of the acetabular ball are spaced from one another, thereby defining a gap therebetween,
a femoral implant for implantation in a medullary canal of a proximal end of a femur,
a femoral cup firmly affixed to a proximal end of the femoral implant, the femoral cup sized for articulation in the gap, such that the femoral cup has a concave surface sized for articulation on the surface of the acetabular ball and a convex surface opposite the concave surface of the femoral cup sized for articulation on the concave surface of the acetabular cup, the gap being sized and configured to permit said articulations while constraining the femoral cup within the gap throughout an entire range of said articulations of the femoral cup as it articulates within the gap, thereby reducing the risk of dislocation, and
the acetabular cup has a notch in the circumferential outer edge thereof to permit maximum inflection when the prosthesis is implanted in a patent.

2. The prosthesis of claim 1 wherein the femoral cup further comprises a notch in the circumferential outer edge thereof.

3. The prosthesis of claim 1 wherein the concave surface of the acetabular cup has a center and the acetabular cup stem is affixed to and in the center.

4. The prosthesis of claim 1 wherein the acetabular ball has an acetabular ball recess sized to receive the acetabular cup stem.

5. The prosthesis of claim 1 wherein the femoral cup has a femoral cup stem projecting outwardly therefrom in a direction opposite the concave surface thereof and the femoral implant has at its proximal end a recess sized to receive the femoral cup stem.

6. The prosthesis of claim 1 wherein the femoral implant has a femoral implant stem extending proximally therefrom and the femoral cup has a recess sized to receive the femoral implant stem.

7. The prosthesis of claim 4 wherein the acetabular ball has a center, the acetabular cup stem has a longitudinal center line and the acetabular ball recess has a longitudinal center line, both longitudinal center lines being colinear and passing through the center of the acetabular ball.

8. The prosthesis of claim 5 wherein the femoral cup has a concave hemispherical portion having a center line, the femoral cup stern has a longitudinal center line and the femoral implant recess has a longitudinal center line wherein all of the center ones are colinear.

9. The prosthesis of claim 1 wherein the concave surface of the femoral cup is fully in contact with the surface of the acetabular ball during articulation of said concave surface of the femoral cup on the acetabular ball.

10. The prosthesis of claim 1 further comprising an articulating protective sheath disposed between the concave surface of the acetabular cup and the convex surface of the femoral cup.

11. A method of implanting in a patient the prosthesis of claim 1 comprising preparing an acetabular socket in a pelvis and affixing the acetabular cup therein, preparing a medullary canal at the proximal end of a femur and affixing the femoral implant therein, affixing the acetabular ball to the acetabular cup stem, affixing the femoral cup to the proximal end of the femoral implant and aligning the concave surface of the femoral cup with the acetabular ball so that the femoral cup will articulate in the gap and the concave surface of the femoral cup will articulate on the surface of the acetabular ball.

12. The method of claim 11 further comprising disposing a protective shield in the acetabular cup prior to affixing the acetabular ball to the acetabular cup stem.

13. A revision surgery method of implanting in a patient the prosthesis of claim 1, the patient having an implant in need of revision which comprises a hip joint prosthesis with an implanted acetabular cup and a femoral stem having an articulating femoral ball affixed to the proximal end thereof, the implanted acetabular cup having a concave articulating surface, the method comprising
removing the femoral stern and articulating femoral ball,
affixing to the concave articulating surface of the implanted acetabular cup the acetabular cup of claim 1,
preparing a medullary canal at the proximal end of a femur and affixing the femoral implant therein, affixing the acetabular ball to the acetabular cup stem, affixing the femoral cup to the proximal end of the femoral implant and aligning the concave surface of the femoral cup with the acetabular ball so that the concave surface of the femoral cup will articulate on the acetabular ball.

14. The method of claim 11 further comprising disposing a protective shield in the acetabular cup prior to affixing the acetabular ball to the acetabular cup stem.

15. A kit comprising the prosthesis of claim 5 and further comprising one or more additional femoral cups, wherein the femoral cup stems have different lengths.

16. A kit comprising the prosthesis of claim 6 wherein the femoral cup recess is disposed in a neck projecting outwardly from the femoral cup in a direction opposite the concave surface thereof, further comprising one or more additional femoral cups, wherein the femoral cup necks have different lengths.

17. A reverse shoulder prosthesis comprising
a unitary glenoid cup having an outer non-articulating surface adapted for attachment to a glenoid fossa and a concave surface located opposite to the non-articulating surface, the concave surface having a glenoid cup stem firmly affixed therein and projecting outwardly therefrom,
a glenoid ball firmly affixed to the glenoid cup stem, the glenoid ball having a surface,
the concave surface of the glenoid cup and the surface of the glenoid ball are spaced from one another, thereby defining a gap therebetween,
a humeral stem for implantation in a medullary canal of a proximal end of a humerus, and
a humeral cup firmly affixed to a proximal end of the humeral stem, the humeral cup sized for articulation in the gap, such that the humeral cup has a concave surface sized for articulation on the surface of the glenoid ball and a convex surface opposite the concave surface of the humeral cup sized for articulation on the concave surface of the glenoid cup, the gap being sized and configured to permit said articulations while constraining the humeral cup within the gap throughout an entire range of said articulations of the humeral cup as it articulates within the gap, thereby reducing the risk of dislocation.

18. The prosthesis of claim 17 wherein the concave surface of the glenoid cup has a center and the glenoid cup stem is affixed thereto and in the center.

19. The prosthesis of claim 17 wherein the glenoid ball has a glenoid ball recess sized to receive the glenoid cup stem.

20. The prosthesis of claim 17 wherein the humeral cup has a humeral cup stem projecting outwardly therefrom in a direction opposite the concave surface thereof and the humeral stem has at its proximal end a recess sized to receive the humeral cup stem.

21. The prosthesis of claim 17 wherein the humeral implant has a humeral implant stem extending proximally therefrom and the humeral cup has a recess sized to receive the humeral implant stem.

22. The prosthesis of claim 19 wherein the glenoid ball has a center, the glenoid cup stem has a longitudinal center line and the glenoid ball recess has a longitudinal center one, both longitudinal center lines being colinear and passing through the center of the glenoid ball.

23. The prosthesis of claim 20 wherein the humeral cup has a concave hemispherical portion having a center line, the humeral cup stem has a longitudinal center line and the humeral implant recess has a longitudinal center line wherein all of the center lines are colinear.

24. The prosthesis of claim 17 wherein the concave surface of the humeral cup is fully in contact with the surface of the glenoid ball during articulation of said concave surface of the humeral cup on the glenoid ball.

25. The prosthesis of claim 17 further comprising an articulating sheath disposed between the concave surface of the glenoid cup and the convex surface of the humeral cup.

26. The prosthesis of claim 17 further comprising a notch in the circumferential outer edge of the glenoid cup.

27. The prosthesis of claim 26 wherein the humeral cup has a notch in the circumferential outer edge thereof.

28. A method of implanting in a patient the prosthesis of claim 17 comprising preparing a glenoid fossa and affixing the glenoid cup therein, preparing a medullary canal at the proximal end of a humerus and affixing the humeral stem therein, affixing the glenoid ball to the glenoid cup stem, affixing the humeral cup to the proximal end of the humeral stem and aligning the concave surface of the humeral cup with the glenoid ball so that the humeral cup will articulate in the gap and the concave surface of the humeral cup will articulate on the surface of the glenoid ball.

29. The method of claim 28 further comprising disposing a protective shield in the glenoid cup prior to affixing the glenoid ball to the glenoid cup stem.

30. A revision surgery method of implanting in a patient the prosthesis of claim 17, the patient having an implant in need of revision which comprises a shoulder joint prosthesis with an implanted glenoid cup and a humeral stem having an articulating humeral ball affixed to the proximal end thereof, the implanted glenoid cup having a concave articulating surface, the method comprising
removing the humeral stem and articulating humeral ball,
affixing to the concave articulating surface of the implanted glenoid cup the glenoid cup of claim 17,
preparing a medullary canal at the proximal end of a humerus and affixing the humeral implant therein, affixing the glenoid ball to the glenoid cup stem, affixing the humeral cup to the proximal end of the humeral implant and aligning the concave surface of the humeral cup with the glenoid ball so that the concave surface of the humeral cup will articulate on the glenoid ball.

31. The method of claim 28 further comprising disposing a protective shield in the glenoid cup prior to affixing the glenoid ball to the glenoid cup stem.

32. A kit comprising the prosthesis of claim 20 and further comprising one or more additional humeral cups, wherein the humeral cup stems have different lengths.

33. A kit comprising the prosthesis of claim 21 wherein the humeral cup recess is disposed in a neck projecting outwardly from the humeral cup in a direction opposite the concave surface thereof, further comprising one or more additional humeral cups, wherein the humeral cup necks have different lengths.
